(19) United States
(12) Patent Application Publication
(10) Pub. No.: US 2026/0103766 A1
(43) Pub. Date: Apr. 16, 2026

(54) METHODS TO DETECT HAPLOID INDUCED PROGENY

(71) Applicant: Rev Genomics, Inc., Oakland, CA (US)

(72) Inventor: Phillip Adam Conklin, Oakland, CA (US)

(73) Assignee: Rev Genomics, Inc., Oakland, CA (US)

(21) Appl. No.: 19/358,401

(22) Filed: Oct. 14, 2025

Related U.S. Application Data
(60) Provisional application No. 63/707,125, filed on Oct. 14, 2024.

(51) Int. Cl. C12Q 1/6895

(52) U.S. Cl. CPC C12Q 1/6895; C12Q 2600/156

(57) ABSTRACT

Exemplary embodiments disclose methods to detect haploid induced progeny in dioecious plants. These methods compare male donor and female acceptor variants unique to each parent and then asks which of these is inherited by the potential haploid induced progeny in question. The inherited variants are further categorized into heterozygous and homozygous variants and plotted along the length of a reference chromosome to create a visual signal of inheritance. Haploid progeny will have a high band signal for homozygous maternal variants and a very low signal for heterozygous maternal variants consistently across the entire length of the chromosome. Furthermore, exemplary embodiments can identify doubled haploids, which aren’t detected in traditional haploid detection methods such as flow cytometry.

### CROSS-REFERENCE TO RELATED APPLICATIONS

This patent application is related to and claims priority benefit under 35 U.S.C. §119(e), to co-pending and commonly-owned U.S. Provisional Patent Application No. 63/707,125, filed October 14, 2024, entitled “METHODS TO DETECT HAPLOID INDUCED PROGENY”, and listing Phillip A Conklin as inventor, which patent document is incorporated by reference herein in its entirety and for all purposes.

### BACKGROUND

### FIELD OF THE INVENTION

This invention relates to the field of plant biotechnology and plant breeding. The presently disclosed subject matter relates to identifying a homozygous doubled haploid or haploid induced progeny and can be distinguished from a hybrid or self-fertilized progeny.

### BRIEF DESCRIPTION OF THE FIGURES

FIG. 1 Exemplary embodiments of relative signal intensity for a cross that results in typical heterozygous progeny.

FIG. 2 Exemplary embodiments of relative signal intensity for a cross that results in a haploid induced progeny.

FIG. 3a Exemplary embodiments of the maternal parent Sneakerheads and the haploid induced progeny identified with the disclosed invention. Diagram in the lower left corner of the exemplary embodiments shows flow cytometry results to verify ploidy.

FIG. 3b Exemplary embodiments of the maternal parent Horchata and the haploid induced progeny identified with the disclosed invention. Diagram in the lower left corner of the exemplary embodiments shows flow cytometry results to verify ploidy.

FIG. 3c Exemplary embodiments of the maternal parent Pineapple Mac and the haploid induced progeny identified with the disclosed invention. Diagram in the lower left corner of the exemplary embodiments shows flow cytometry results to verify ploidy.

FIG. 3d Exemplary embodiments of the maternal parent Sneakerheads and the haploid induced progeny identified with the disclosed invention. Diagram in the lower left corner of the exemplary embodiments shows flow cytometry results to verify ploidy.

FIG. 3e Exemplary embodiments of the maternal parent Sneakerheads and the haploid induced progeny identified with the disclosed invention. Diagram in the lower left corner of the exemplary embodiments shows flow cytometry results to verify ploidy.

FIG. 4 shows an exemplary method to detect haploid induced progeny.

### DETAILED DESCRIPTION OF EMBODIMENTS

The exemplary embodiments described herein solve a problem of detecting haploids in organisms with poorly established reference genomes, or sequenced individuals with high levels of somatic mutations, or spontaneously doubled haploids that would otherwise be missed as false negatives with traditional methods like flow cytometry. Even after filtering low quality SNPs, there are a large number of false or non-germ line sequence variants or mis mapped multi allelic regions. By first removing all common sequence variants between parents and evaluating only sequence variants inherited from the mom and/or dad, we are conservatively evaluating mostly germ-line transmissible variants. Variants meaning SNPs (Single nucleotide polymorphism) that vary at a single base position along the reference genome.

There are various methods to generating a haploid progeny, some may include a cross with a male and female, others that only use one parent’s gamete precursors. Here we have an example using a cross between a male and female, which results in lots of offspring. Some of the offspring will be normal diploid individuals, and some will be haploid individuals. This method sequences all individuals involved from the parents to the offspring to filter unwanted or misleading variants to determine whether the progeny is haploid/doubled haploid or a normal diploid individual with heterozygous loci resulting from genetic information contributed by both parents, which unless you are selfing a homozygous parent, will result in a heterozygous diploid offspring.

Haploids are used in plant breeding to accelerate the generation of phenotypically stable F1 hybrid seed. Haploids can be generated in myriad ways, but a critical step in the process is the identification of the said haploid induced progeny. In corn the Mangelsdorf tester (Mangelsdorf 1948), which has a visual phenotypic marker linked to each chromosome, can be used to identify haploid induced progeny. Understanding the chromosomal inheritance is fundamental to the identification process. Modern sequencing technology allows us to investigate all genomic inheritance as shown in the disclosed invention. Here we use Cannabis as a model for inducing and identifying haploids in dioecious crops.

Cannabis sativa is a dioecious crop desired for its narcotic, medical, fiber and biofuel properties. To date, propagation of consistent Cannabis varieties relies on clonal propagation. However, this requires ample space for the “mother plants”, high shipping and handling costs, and is left susceptible to transmissible pests and diseases. To avoid these costs and risks, seeds have been choice for many crops. Phenotypically stable F1 hybrid seed requires the use of inbred parents via many rounds of self-fertilization or within one generation via haploid inducer technology. Currently, the genetics of Cannabis seed is much too variable for cultivators to achieve a consistent crop, thus the need for a method to detect haploid induced progeny.

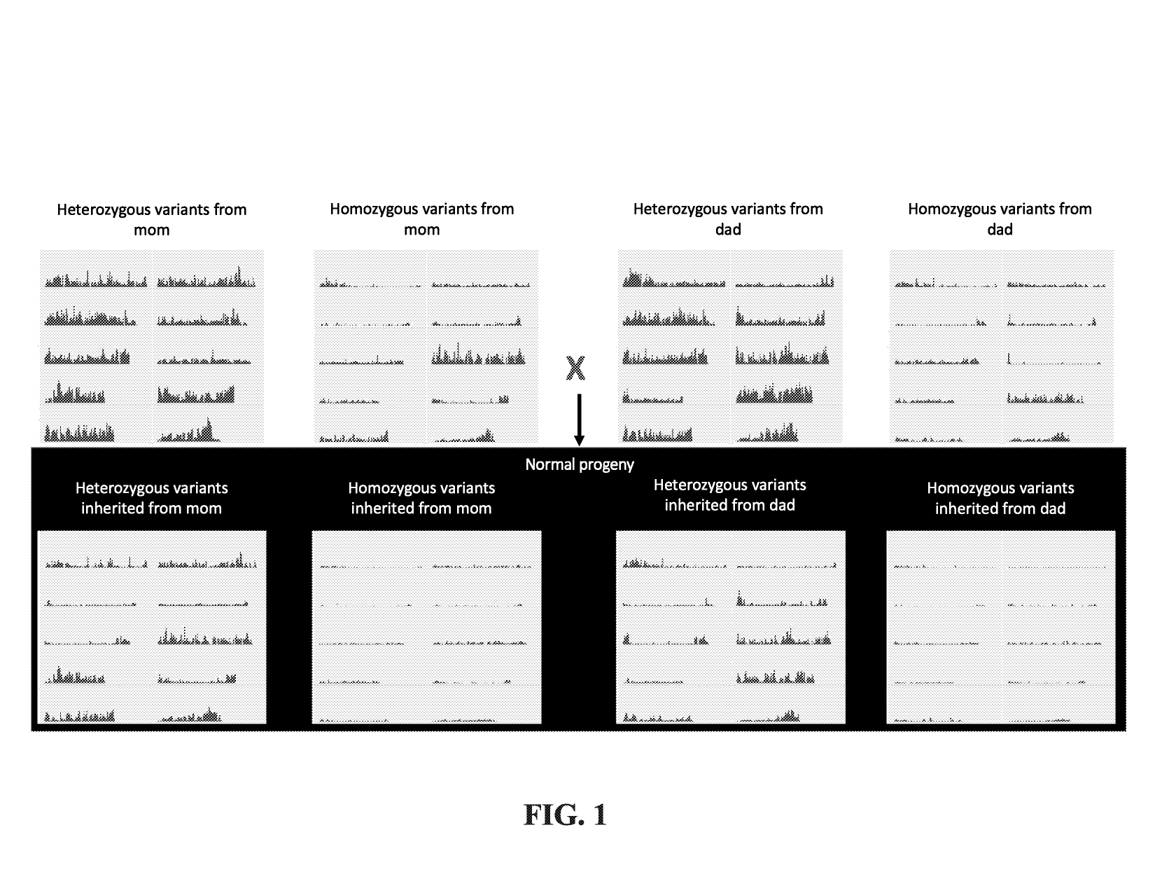

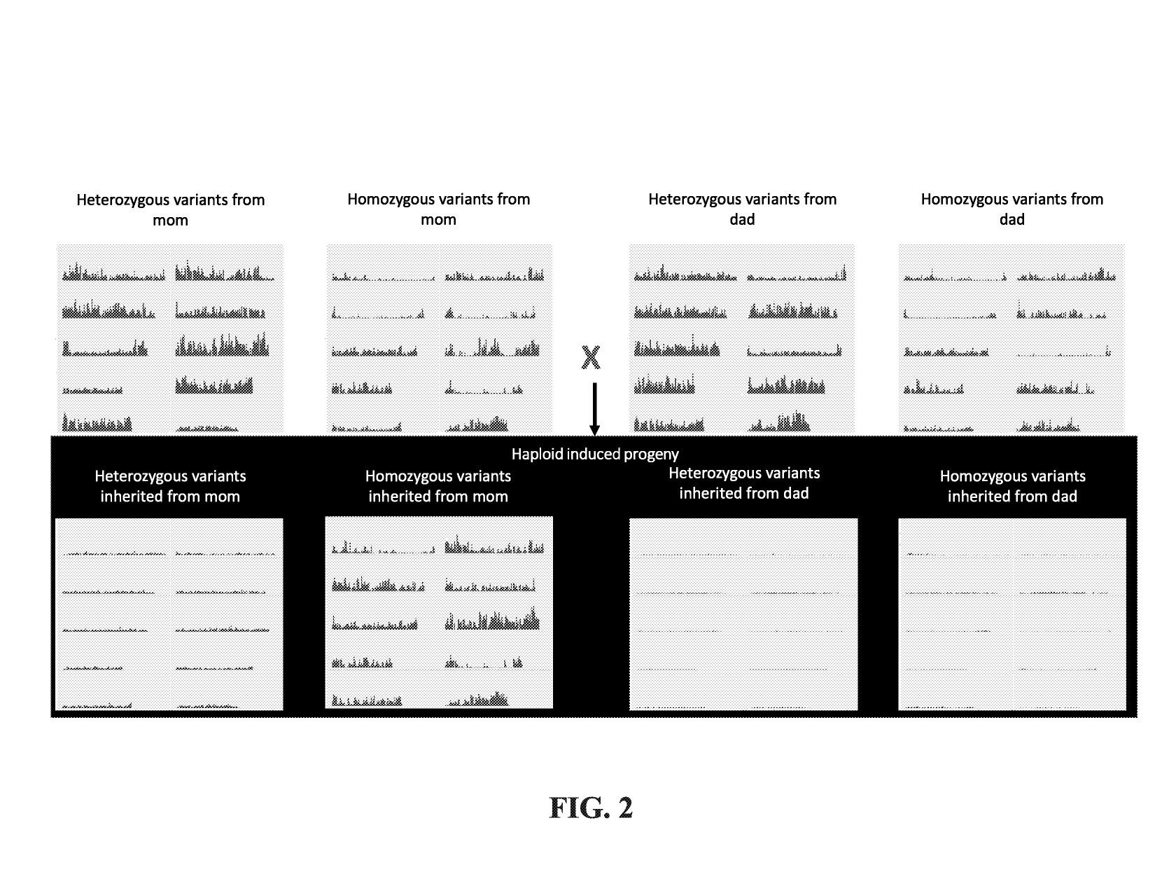

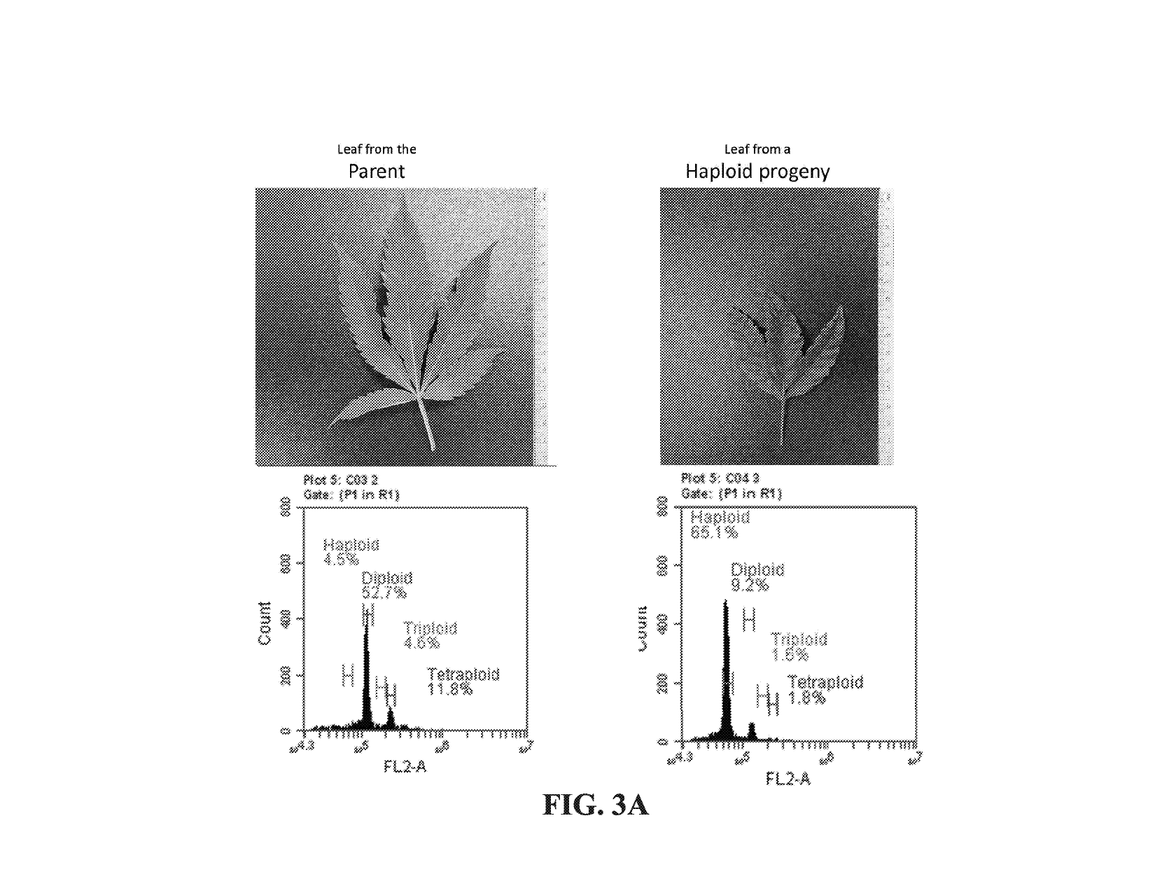

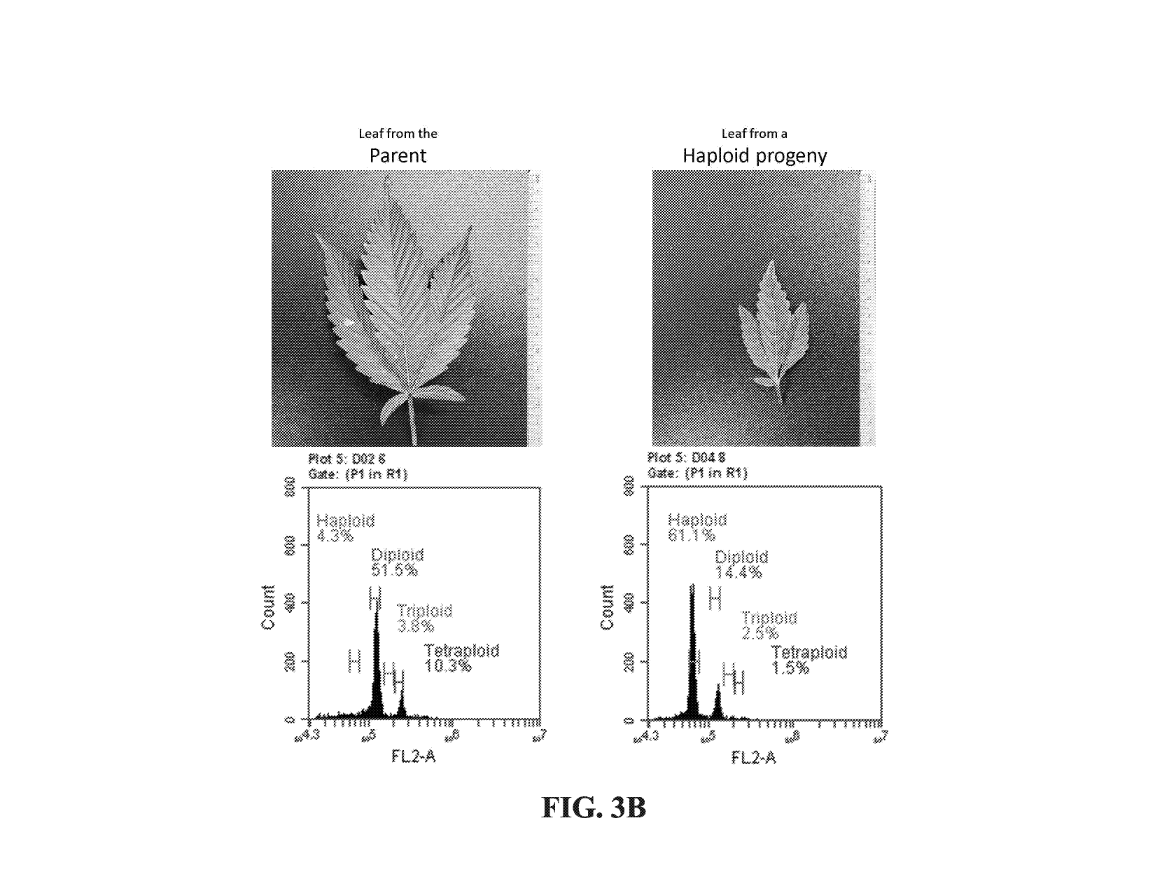

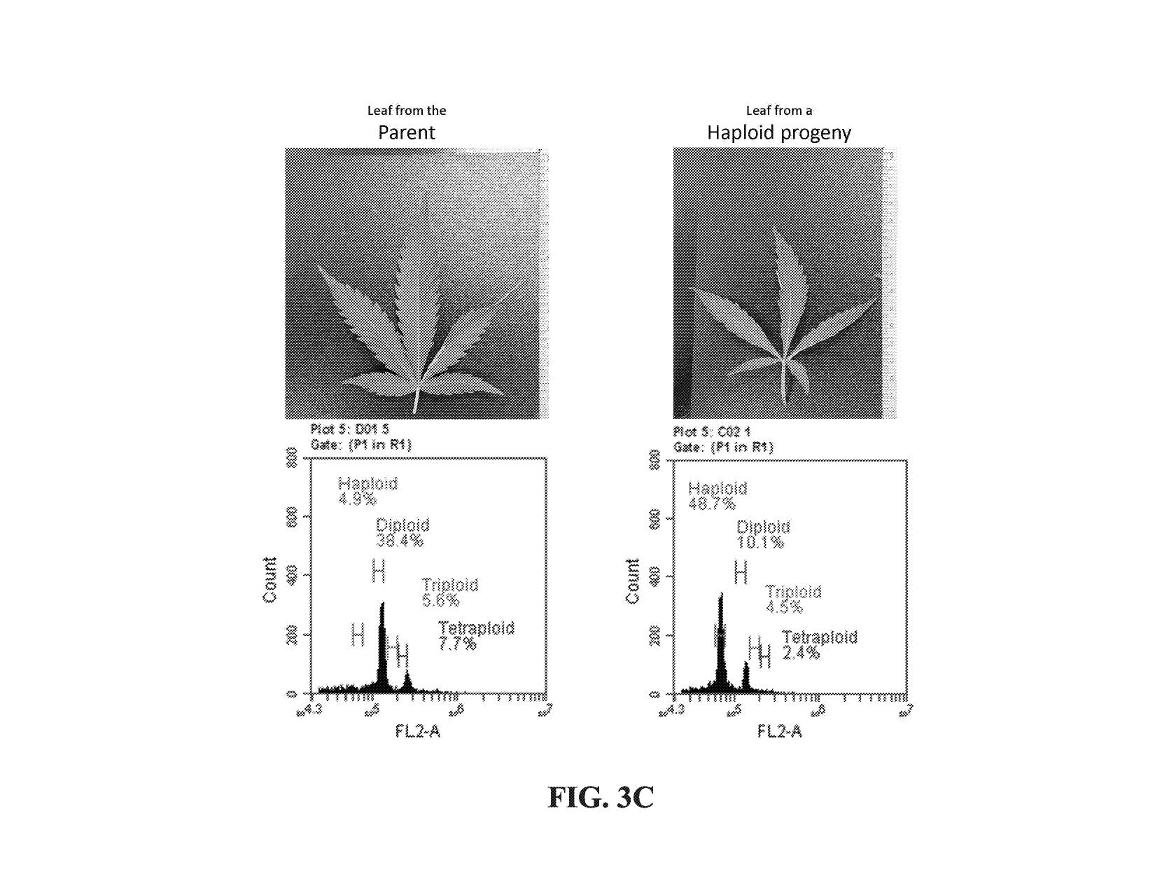

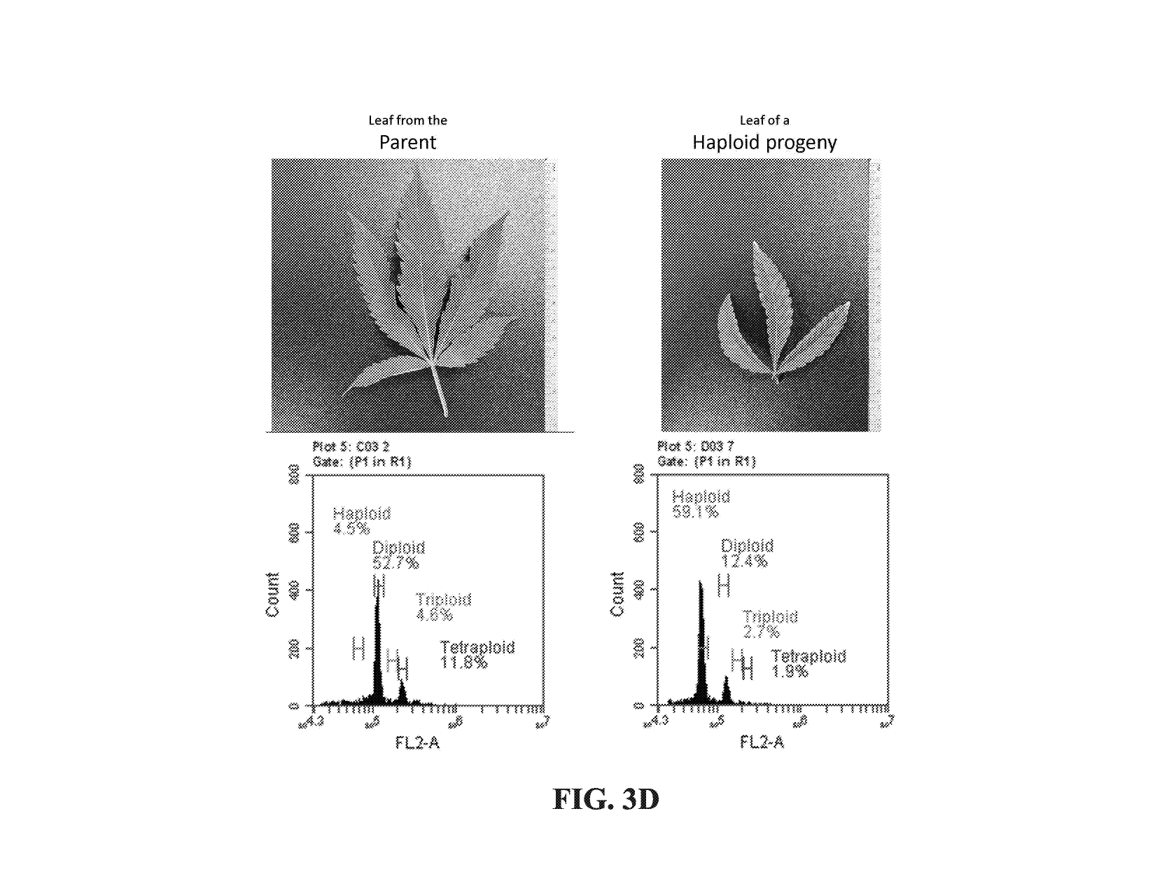

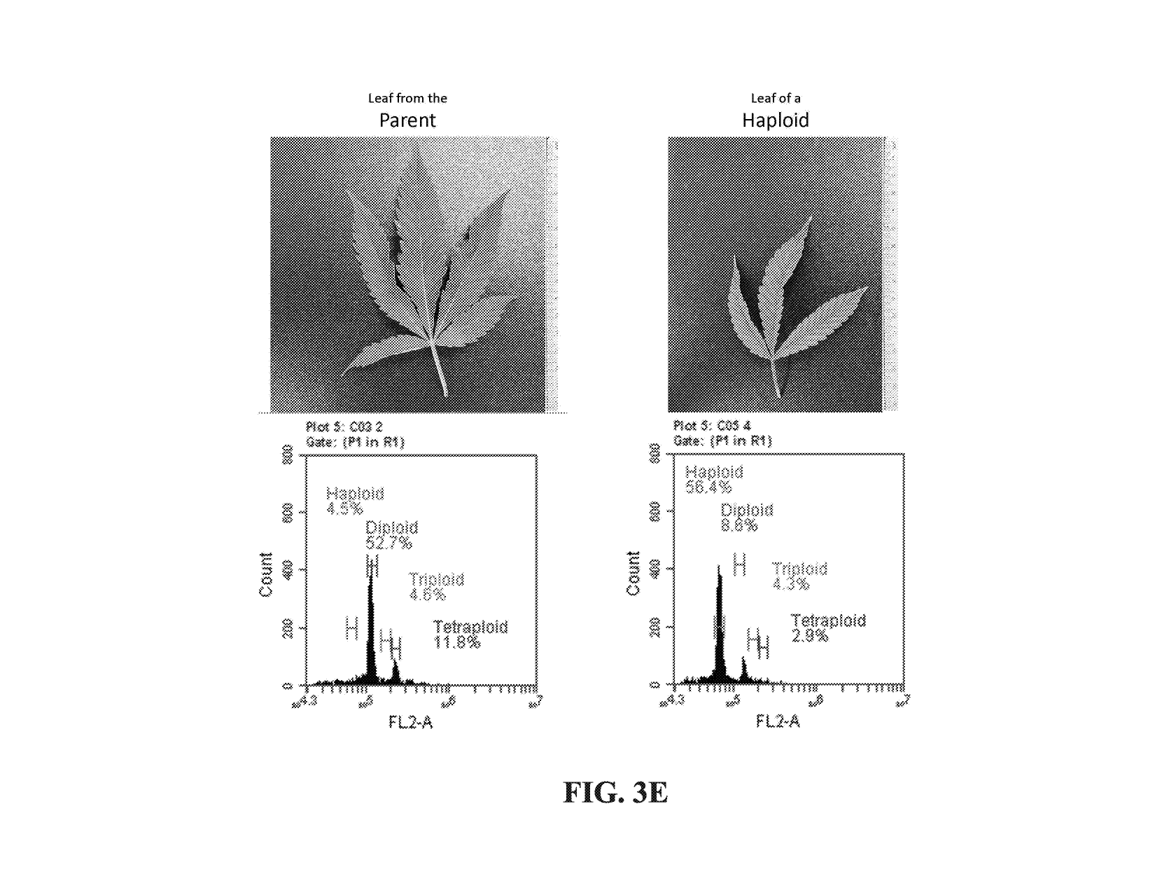

Exemplary embodiments make use of DNA sequences with as little as 10x coverage mapped to a reference genome to identify haploid induced progeny. Intersecting variants of each parent yield unique variants to the mother and father of the haploid induced progeny. Further intersecting these unique variants to the progeny will identify the variants that are inherited from the respective parents. Plotting the inherited maternal or paternal variants that categorize as either homozygous or heterozygous along the length of the 10 chromosomes of Cannabis can create a visual signal with an intensity relative to the frequency of inheritance for each category. These signals are like bands on an electrophoresis gel, in that the intensity and clarity of the visual signal is the interpretable data. FIG. 1 shows the resulting breakdown of a normal cross between two heterozygous diploid parents. In this case the heritable signal in the progeny is mostly categorized in the heterozygous state with roughly equal representation from either parent. FIG. 2 diagrams a cross that results in a haploid induced progeny, which shows the signal nearly entirely found in the homozygous category inherited exclusively from the maternal parent. This was the visual signal observed in each case for confirmed haploid induced progeny as exemplified in FIGS. 3a-e.

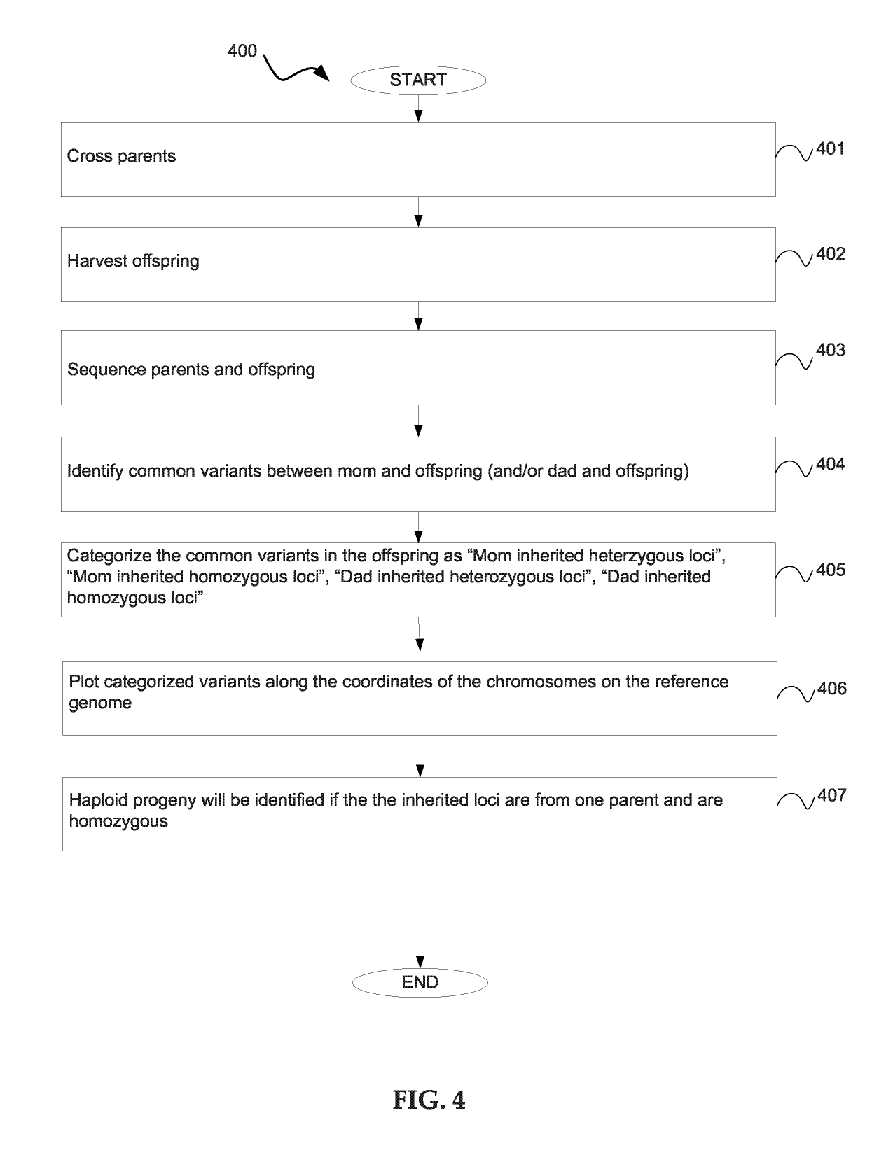

FIG. 4 shows an exemplary method to detect haploid induced progeny.

At step 401, the parents are crossed.

At step 402, the offspring is harvested.

At step 403, the parents and offspring are sequenced.

At step 404, common variants between mom and offspring are identified and common variants between dad and offspring are identified.

At step 405, the common variants are categorized as: “Mom inherited heterzygous loci”, “Mom inherited homozygous loci”, and/or “Dad inherited heterozygous loci”, “Dad inherited homozygous loci”.

At step 406, the categorized variants are plotted along the coordinates of the chromosomes on the reference genome.

At step 407, haploid progeny are identified if the inherited loci are from one parent and are homozygous.

### Works cited

Abe, A., Kosugi, S., Yoshida, K. et al. Genome sequencing reveals agronomically important loci in rice using MutMap. Nat Biotechnol 30, 174–178 (2012). https://doi.org/10.1038/nbt.2095

Henry IM, Dilkes BP, Miller ES, Burkart-Waco D, Comai L. Phenotypic consequences of aneuploidy in Arabidopsis thaliana. Genetics. 2010 Dec;186(4):1231-45. doi: 10.1534/genetics.110.121079. Epub 2010 Sep 27. PMID: 20876566; PMCID: PMC2998307.

Klein, H., Xiao, Y., Conklin, P. A., Govindarajulu, R., Kelly, J. A., Scanlon, M. J., ... & Bartlett, M. (2018). Bulked-segregant analysis coupled to whole genome sequencing (BSA-Seq) for rapid gene cloning in maize. G3: Genes, Genomes, Genetics, 8(11), 3583-3592.

Mangelsdorf P.C. (1948) Multiple gene linkage testers. Maize Genet. Coop. Newsl. 22, 22.

What is claimed is:

1. A claim where a homozygous plant is identified using nucleotide sequence variants to interpret zygosity of inherited maternally or paternally derived variants.
a. A method of claim one where a homozygous plant is either haploid or doubled- haploid.
b. A method of claim one where nucleotide sequence variants distributed along the reference genome chromosomes are used to generate a signal for relative intensity of inherited maternally or paternally derived homozygous and heterozygous variants.
c. A method of claim one where the possibility of a hybrid, aneuploid, pollen contamination, or hermaphroditic self-fertilization is identified.
